(19) United States
(12) Patent Application Publication
(10) Pub. No.: US 2006/0210515 A1
(43) Pub. Date: Sep. 21, 2006

(54) HAIR GROWTH FORMULA

(76) Inventor: Thomas E. Mower, Payson, UT (US)

Correspondence Address: STARKWEATHER & ASSOCIATES, 9035 SOUTH 1300 EAST, SUITE 200, SANDY, UT, 84094, US

(21) Appl. No.: 11/306,998

(22) Filed: Jan. 18, 2006

Related U.S. Application Data
(63) Continuation-in-part of application No. 11/083,826, filed on Mar. 18, 2005.

(51) Int. Cl. A61K 8/73; A61K 31/737

(52) U.S. Cl. 424/70.13; 424/70.9; 514/54

(57) ABSTRACT

A hair treatment composition for topical application to the skin and/or hair, comprising partially hydrolyzed fucoidan and a pharmaceutical carrier. The composition may contain further additives. The carrier may be hydrophyllic or anhydrous. The composition may also include natural components such as honey and/or mangosteen. The composition may also include flavonoids, analgesics, radiation protecting agents, and/or anti-oxidants.

This application is a Continuation-in-Part of, and claims the benefit of application Ser. No. 11/083,826, filed on 18 Mar. 2005, by Thomas E. Mower, entitled Fucoidan Compositions and Methods for Dietary and Nutritional Supplements, the entirety of which is herein incorporated by reference.

### BACKGROUND OF THE INVENTION

1. Field of the Invention

The present invention relates generally to hair growth formulas and, more particularly, to hair growth formulas for topical application.

2. Description of the Related Art

The loss of hair is a continuing problem among both men and women. Though individual hairs typically live, die, and are replaced, at times, the hairs die prematurely, and/or are not replaced. As a result, a net loss of hair is experienced. This net loss of hair is often perceived as a detriment. For these reasons, consumers continue to search for compositions for slowing the loss of hair, and/or stimulating the growth of hair.

Human hair has a three-phase life cycle, namely the anagen phase, the catagen phase, and the telogen phase. In the anagen phase is the phase wherein the hair grows longer. The anagen phase may last for several years. The phases last for different periods of time, depending on what part of the body the hair grows. On the scalp, for example, the anagen phase may last for 2-3 years, or even longer. The transitory catagen phase follows, and is typically much shorter than the anagen phase. The catagen phase may last about 2 to 3 weeks on the scalp. Finally, the hair enters into the telogen phase. The telogen phase is the resting phase, after which the hair falls out. The telogen phase is also typically shorter than the anagen phase. The telogen phase typically lasts about 3 months on the scalp. Typically, following the telogen phase, a new hair begins to grow, thus beginning again the three-stage cycle of the hair.

Though there are many causes of hair loss, in a large number of cases, the premature loss of the hair occurs in genetically predisposed subjects and it affects men in particular. It involves more particularly androgenetic, androgenic or alternatively androgeno-genetic alopecia.

This alopecia is essentially due to a disruption in hair renewal which causes, in the first instance, the acceleration of the frequency of the cycles at the expense of the quality of the hair and then of its quantity. A gradual deterioration of the hair is brought about by regression of the hair. Some areas are typically affected, in particular the temporal or frontal sinuses and the upper part of the occipital in men, whereas in women a diffuse alopecia of the vertex is mainly observed.

One result of alopecia is the formation of weaker, thinner hairs called vellus hairs. Terminal hairs are coarse, pigmented, long hairs in which the bulb of the hair follicle is seated deep in the dermis. Vellus hairs, however, are fine, thin, non-pigmented short hairs in which the hair bulb is located superifically in the dermis. As alopecia progresses, a transition takes place in the area of approaching baldness wherein the hairs themselves are changing from the terminal to the vellus type. As more hairs change from the terminal to the vellus type, the area where the changes occur appears to be bald due to the skimpiness of the vellus hairs.

Many drug and non-drug approaches have been taken to slow the changes from terminal to vellus hairs, replace hairs, excite the growth of terminal hairs, and so forth. By far, the most common approach to the problem of discovering a remedy for male pattern alopecia has been one of drug therapy. Many types of drugs ranging from vitamins to hormones have been tried and only recently has there been any indication whatsoever of even moderate success. For instance, it was felt for a long time that since an androgenic hormone was necessary for the development of male pattern baldness, that either systemic or topical application of an antiandrogenic hormone would provide the necessary inhibiting action to keep the baldness from occurring. The theory was promising but the results were uniformly disappointing.

The androgenic hormone testosterone was known, for example, to stimulate hair growth when applied topically to the deltoid area as well as when injected into the beard and pubic regions. Even oral administration was found to result in an increased hair growth in the beard and pubic areas as well as upon the trunk and extremities. While topical application to the arm causes increased hair growth, it is ineffective on the scalp and some thinning may even result. Heavy doses of testosterone have even been known to cause male pattern alopecia.

Certain therapeutic agents have been known to induce hair growth in extensive areas of the trunk, limbs and even occasionally on the face. Such hair is of intermediate status in that it is coarser than vellus but not as coarse as terminal hair. The hair is generally quite short with a length of 3 cm. being about maximum. Once the patient ceases taking the drug, the hair reverts to whatever is normal for the particular site after six months to a year has elapsed. An example of such a drug is diphenylhydantoin which is an anticonvulescent drug widely used to control epileptic seizures. Hypertrichosis (excessive hair growth) is frequently observed in epileptic children some two or three months after starting the drug and first becomes noticeable on the extensor aspects of the limbs and later on the trunk and face. The pattern is not unlike that sometimes caused by injury to the head. As for the hair, it is often shed when the drug is discontinued but may, in some cicumstances, remain.

Streptomycin is another drug that has been found to produce hypertrichosis in much the same way as diphenylhydantoin when administered to children suffering from tuberculous meningitis. About the same effects were observed and the onset and reversal of the hypertrichosis in relation to the period of treatment with the antibiotic leave little question but that it was the causative agent.

Transplantation is another known response to hair loss. Plugs of skin containing hair are transplanted from areas of the scalp where hair is growing to bald areas with reasonable success; however, the procedure is a costly one in addition to being time-consuming and quite painful. Furthermore, the solution is inadequate from the standpoint that it becomes a practical, if not an economic, impossibility to replace but a tiny fraction of the hair present in a normal healthy head of hair.

One example is disclosed by Chidsey III, in U.S. Pat. No. 4,569,812, which discloses the compound 6-amino-1,2-dihydro-1-hydroxy-2-imino-4-piperidinopyrimidine for use as a therapeutic agent to arrest and reverse male pattern alopecia. The compound itself was discovered by William C. Anthony and Joseph J. Ursprung and it forms the subject matter among other similar compounds of U.S. Pat. No. 3,461,461 issued Aug. 12, 1969. This compound, among others, may have considerable therapeutic value in the treatment of severe hypertension. It is a so-called “vasodilator” which, as the name implies, functions to dilate the peripheral vascular system.

Vasodilators as a general class of therapeutic agents typically do not prove effective to grow hair on the scalp as a result of topical application thereof to bald areas. According to the U.S. Pat. No. 4,569,812 patent, male pattern alopecia can be treated by repeated topical application of a composition containing as one of its active ingredients 6-amino-1,2-dihydro-1-hydroxy-2-imino-4-piperodinopyrimidine, hereinafter to be referred to by the coined term “Minoxidil” to affected areas of the human scalp.

As disclosed in U.S. Pat. No. 3,461,461, Minoxidil comprises a compound of the formula: 6-amino-1,2-dihydro-1-hydroxy-2-iminopyrimidines, their carboxyacylated counterparts, and the corresponding acid addition salts thereof are disclosed. The compounds, useful inter alia as antihypertensive agents, are substituted in the 4-position and optionally in the 5-position, the substituent in the 4-position being a secondary or tertiary amino moiety.

In another example of disclosures of formulas to combat hair loss, Chidsey, III discloses in U.S. Pat. No. 4,139,619, a process for stimulating the growth of mammalian hair comprising the application to mammalian skin of a compound of the formula: 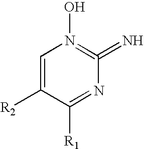
wherein R₁ is a moiety selected from the group consisting of moieties of the formula 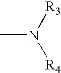
wherein R₁ and R₁ are selected from the group consisting of hydrogen, lower alkyl, lower alkenyl, lower aralkyl, and lower cycloalkyl, and taken together R₁ and R₁ may be a heterocyclic moiety selected from the group consisting of aziridinyl, azetidinyl, pyrrolidinyl, piperidino, hexahydroazepinyl, heptamethylenimino, octamethylenimino, morpholino, and 4-lower-alkylpiperazinyl, each of said heterocyclic moieties having attached as substituents on the carbon atoms 0-3 lower alkyl groups, hydroxy or alkoxy wherein R₁ is selected from the group consisting of hydrogen, lower alkyl, lower alkenyl, lower alkoxyalkyl, lower cycloalkyl, lower aryl, lower aralkyl, lower alkaryl, lower alkaralkyl, lower alkoxyaralkyl, and lower haloaralkyl and the pharmacologically acceptable acid addition salts thereof in association with a topical pharmaceutical carrier.

Further, in U.S. Pat. No. 6,602,869, which is herein incorporated by reference, Galey discloses compounds of formula (I) wherein R₁ represents a polymer, aryl or alkyl group, R₂ represents a hydrogen atom, a halogen atom, a radical CN, CF₃, OH, OCF₃, COOH, R⁷, OR⁷ or OCOR⁷, R³ represents an aryl or alkyl halogen, R₄ represents an alkyl radical. The invention also relates to a cosmetic composition comprising said compounds and to the use thereof to reduce and/or curb hair loss i.e. to increase and/or stimulate the growth of said hair. Formula (I) is as follows:

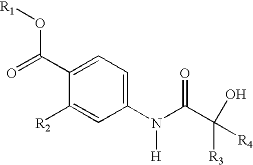

Consumer demand for natural-based products has been growing in recent years. Chemical synthesis is perceived as environmentally unsafe. A chemically synthesized component may contain harsh chemicals. Natural products are perceived as more pure and mild, and thus superior to chemically synthesized products. Delivering a cosmetic benefit from plant sources, however, is not trivial. To derive a real benefit from a natural source, not only does a plant or a part of the plant containing a specific active component have to be identified, but a minimum concentration and/or a specific extract of that plant has to be identified that truly delivers a benefit.

Accordingly, consumers demand an effective hair treatment composition that moisturizes the skin, heals, and soothes the vulnerable and delicate surface of the skin, as well as slowing the loss of hair and/or promoting the growth of hair. Further, consumers demand that treatment for the skin include natural products to promote healing and preserve youthful appearance.

Fucoidan is a sulfated polysaccharide found in many sea plants and animals, and is particularly concentrated in the cell walls of brown algae (Phaeophyceae). Fucoidan is a complex carbohydrate polymer composed mostly of sulfated L-fucose residues. These polysaccharides are easily extracted from the cell wall of brown algae with hot water or dilute acid and may account for more than 40% of the dry weight of isolated cell walls. O. Berteau & B. Mulloy, Sulfated Fucans, Fresh Perspectives: Structures, Functions, and Biological Properties of Sulfated Fucans and an Overview of Enzymes Active Toward this Class of Polysaccharide, 13 Glycobiology 29R-40R (2003). Fucoidan structure appears to be linked to algal species, but there is insufficient evidence to establish any systematic correspondence between structure and algal order. High amounts of α(1-3) and α(1-4) glycosidic bonds occur in fucoidans from Ascophyllum nodosum. A disaccharide repeating unit of alternating α(1-3) and α(1-4) bonds represents the most abundant structural feature of fucoidans from both A. nodosum and Fucus vesiculosus which are specific species of sea weed. Sulfate residues are found mainly in position 4. Further heterogeneity is added by the presence of acetyl groups coupled to oxygen atoms and branches, which are present in all the plant fucoidans. Following is a representation of A. nodosum fucoidan:

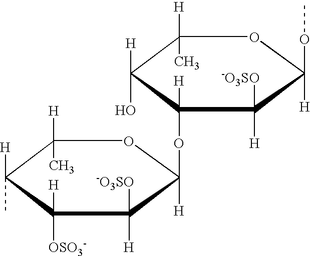

Fucoidan-containing seaweeds have been eaten and used medicinally for at least 3000 years in Tonga and at least 2000 years in China. An enormous amount of research has been reported in the modern scientific literature, where more than 500 studies are referenced in a PubMed search for fucoidan.

The physiological properties of fucoidans in the algae appear to be a role in cell wall organization and possibly in cross-linking of alginate and cellulose and morphogenesis of algal embryos. Fucoidans also have a wide spectrum of activity in biological systems. They have anticoagulant and antithrombotic activity, act on the inflammation and immune systems, have antiproliferative and antiadhesive effects on cells, and have been found to protect cells from viral infection.

Further, fucoidan has numerous beneficial functions that heal and strengthen different systems of the body, including anti-viral, anti-inflammatory, anti-coagulant, and anti-tumor properties. A. I. Usov et al., Polysaccharides of Algae: Polysaccharide Composition of Several Brown Algae from Kamchatka, 27 Russian J. Bio. Chem. 395-399 (2001). Fucoidan has been found to build and stimulate the immune system. Research has also indicated that fucoidan reduces allergies, inhibits blood clotting, fights diabetes by controlling blood sugar, prevents ulcers, relieves stomach disorders, reduces inflammation, protects the kidneys by increasing renal blood flow, and detoxifies the body. Fucoidan also helps to reduce and prevent cardiovascular disease by lowering high cholesterol levels and activating enzymes involved in the beta-oxidation of fatty acids.

Fucoidan has been touted as helping with hair growth following chemotherapy or radiation therapy. Rita Elkins discloses this notion in Limu Moui, Prize Sea Plant of Tonga and the South Pacific, 2001 Woodland Publishing, Pleasant Grove, Utah. This disclosure states that the limu moui can be taken internally or externally in “poultices, gels, and compresses.”

A Japanese study found that fucoidans enhanced phagocytosis, the process in which white blood cells engulf, kill, digest, and eliminate debris, viruses, and bacteria. An American study reported that fucoidans increased the number of circulating mature white blood cells. An Argentine study and a Japanese study found that fucoidans inhibited viruses, such as herpes simplex type I, from attaching to, penetrating, and replicating in host cells. A Swedish study is among the many that showed fucoidans inhibit inflammation cascades and tissue damage that may lead to allergies. Other studies, such as one in Canada, found that fucoidans block the complement activation process that is believed to play an adverse role in chronic degenerative diseases, such as atherosclerosis, heart attack, and Alzheimer's disease. Two American studies found that fucoidans increase and mobilize stem cells.

Researchers have also determined that fucoidan tends to combat cancer by reducing angiogenesis (blood vessel growth), inhibiting metastasis (spreading of cancer cells to other parts of the body), and promoting death of cancer cells. Certain societies that make brown seaweed part of their diet appear to have remarkably low instances of cancer. For example, the cancer death rate in Okinawa is the lowest of all the prefectures in Japan. It is noteworthy that the prefecture of Okinawa, where the inhabitants enjoy some of the highest life expectancies in Japan, also happens to have one of the highest per capita consumption rates of fucoidans.

Brown seaweed, a ready source of fucoidan, is found in abundance in various ocean areas of the world. One of the best locations that provides some of the highest yields of fucoidan is in the clear waters surrounding the Tongan islands, where the seaweed is called limu moui. In Japan, hoku kombu (Laminaria japonica), is said to be particularly rich in fucoidans and is similar to limu moui. The Japanese also consume at least two other types of brown seaweed-wakame and mozuku (Cladosiphon and Nemacystus).

Typically, about four percent by weight of Tongan limu moui is fucoidan. There are at least three types of fucoidan polymer molecules found in brown seaweed. U-fucoidan, having about 20 percent glucuronic acid, is particularly active in carrying out cancer cell destruction. F-fucoidan, a polymer of mostly sulfated fucose, and G-fucoidan, which contains galactose, both tend to induce the production of HGF cells that assist in restoring and repairing damaged cells. All three types of fucoidan also tend to induce the production of agents that strengthen the immune system.

What is needed is a hair growth formula that solves one or more of the problems described herein and/or one or more problems that may come to the attention of one skilled in the art upon becoming familiar with this specification. One problem not solved by the cited art is a hair growth formula that assists in the regeneration of skin. A further problem not solved by the cited art is use of a natural ingredient to assist in hair growth. Yet a further problem includes a hair growth formula that also assists in providing for anti-aging, and regeneration of cells and tissues, promoting youthfulness, reducing inflammation, minimizing the visible signs of biological and/or environmental aging, and/or is high in antioxidants to assist in fighting free radicals.

### SUMMARY OF THE INVENTION

The present invention has been developed in response to the present state of the art, and in particular, in response to the problems and needs in the art that have not yet been fully solved by currently available hair treatment compositions. According to one embodiment of the present invention is a hair growth formula for topical application to the skin and/or hair, comprising partially hydrolyzed fucoidan and a pharmaceutical carrier.

The partially hydrolyzed fucoidan may be derived from the group consisting of: Japanese mozuku seaweed, Japanese kombu seaweed, Tongan limu moui seaweed, and combinations thereof. The partially hydrolyzed fucoidan may be sulfonated. The partially hydrolyzed fucoidan may be dehydrated.

According to one embodiment, the pharmaceutical carrier may be a powder. The powder may be mica.

According to another embodiment, the composition may further include a radiation protecting agent. The radiation protecting agent may be titanium dioxide.

In yet another embodiment, the composition may further include a hair treatment component selected from the group consisiting of: 6-amino-1,2-dihydro-1-hydroxy-2-imino-4-piperidinopyrimidine, 4-(O-methyloxime)phenyl ester of 2-hydroxy-4-(3,3,3-trifluoro-2-hydroxy-2-methylpropionylamino)benzoic acid; phenyl ester of 2-hydroxy-4-(3,3,3-trifluoro-2-hydroxy-2-methylpropionylamino)benzoic acid; methyl ester of 4-(3,3,3-trifluoro-2-hydroxy-2-methylpropionylamino)benzoic acid; para-tolyl ester of 2-hydroxy-4-(3,3,3-trifluoro-2-hydroxy-2-methylpropionylamino)benzoic acid; phenyl ester of 2-hydroxy-4-(2-phenyl-2-hydroxypropionylamino)benzoic acid; methyl ester of 2-methoxy-4-(3,3,3-trifluoro-2-hydroxy-2-methylpropionylamino)benzoic acid; phenyl ester of 2-chloro-4-(3,3,3-trifluoro-2-hydroxy-2-methylpropionylamino)benzoic acid; methyl ester of 2-ethyl-4-(3,3,3-trifluoro-2-hydroxy-2-methylpropionylamino)benzoic acid; 3,5-bis(trifluoromethyl)benzyl ester of 2-hydroxy-4-(3,3,3-trifluoro-2-hydroxy-2-methylpropionylamino)benzoic acid; tert-butyl ester of 2-hydroxy-4-(3,3,3-trifluoro-2-hydroxy-2-methylpropionylamino)benzoic acid; 3-morpholinopropyl ester of 2-hydroxy-4-(3,3,3-trifluoro-2-hydroxy-2-methylpropionylamino)benzoic acid; 4-octylphenyl ester of 2-hydroxy-4-(3,3,3-trifluoro-2-hydroxy-2-methylpropionylamino)benzoic acid; 2,4-diaminopyrimidine-N-oxide, methyl ester of 2-hydroxy-4-(3,3,3-trifluoro-2-hydroxy-2methylpropionylamino)benzoic acid, ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, 7,9(11)-dien-6-one ecdysteroid, and combinations thereof.

In a further embodiment, the composition may further include an anti-oxidant. The anti-oxidant may include one of the group consisting of: butylated hydroxy benzoic acids, 6-hydroxy-2,5,7,8-tetramethylchroman-2-carboxylic acid, gallic acid, propyl gallate, uric acid, sorbic acid, ascorbyl esters of fatty acids, amines, sulfhydryl compounds, dihydroxy fumaric acid, pharmaceutically acceptable salts thereof, alkyl esters thereof, derivatives thereof and mixtures thereof.

In yet a further embodiment, the composition may further include a flavonoid. The flavonoid may include one selected from the group consisting of: unsubstituted flavanone, mono-hydroxy flavanones, mono-alkoxy flavanones, unsubstituted chalcone, mono-hydroxy chalcones, di-hydroxy chalcones, tri-hydroxy chalcones, unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones, unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

In still a further embodiment, the composition may further include one natural component selected from the group consisting of: witch hazel, mangosteen, honey, aloe, sage, piper, clove, ginger, red pepper, willow, rhubarb, sesame, chamomile, propolis, thyme, lavender, cinnamon oil, flower or blossom oils, olive oil, palm oil, coconut oil, beeswax, and mixtures thereof.

In another further embodiment, the composition may further include from about 0.01 weight percent to about 10 weight percent of a derivative of mangosteen. The composition may further include honey.

In yet another embodiment, the composition may further include an analgesic. The analgesic may be selected from the group consisting of: hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, arncinafide, betamethasone, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, piroxicam, isoxicam, tenoxicam, sudoxicam, CP-14,304, aspirin, disalcid, benorylate, trilisate, safapryn, solprin, diflunisal, and fendosal, diclofenac, fenclofenac, indomethacin, sulindac, tolmetin, isoxepac, furofenac, tiopinac, zidometacin, acematacin, fentiazac, zomepiract, clidanac, oxepinac, and felbinac, mefenamic, meclofenamic, flufenamic, niflumic, and tolfenamic acid, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic, phenybutezone, oxyphenbutezone, feprazone, azapropezone, and trimethazone and mixtures thereof.

In yet another further embodiment, the composition may further include a base. The base may include an aqueous phase, an emulsifying agent, and/or an oleaginous phase.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

In describing and claiming the present invention, the following terminology will be used in accordance with the definitions set out below.

As used herein, “comprising,” “including,” “containing,” “is,” “are,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “partially hydrolyzed fucoidan” means fucoidan that has been hydrolyzed into smaller polymers and oligomers, but not so thoroughly hydrolyzed as to result in complete hydrolysis to substantially primarily monosaccharides.

As used herein, “lotions” are liquids, often suspensions or dispersions, intended for external application to the body.

As used herein, “creams” are soft preparations. Creams of the oil-in-water (O/W) type include preparations such as foundation creams, hand creams, shaving creams, and the like. Creams of the water-in-oil (W/O) type include cold creams, emollient creams, and the like. Pharmaceutically, creams are solid emulsions containing suspensions or solutions of active components for external application. Generally, preparations of this type are classified as ointments. Specifically, they belong to the emulsion-type bases.

As used herein, “ointments” are semisolid preparations for external application of such consistency that may be readily applied to the skin. They should be of such composition that they soften, but not necessarily melt, when applied to the body. They serve as vehicles for the topical application of active components and also function as protectives and emollients for the skin. For many years ointments were limited by definition and use to mixtures of fatty substances. Today, in addition to such oleaginous mixtures, there are ointment preparations possessing the same general consistency but entirely free of oleaginous substances. In many instances, they are emulsions of fatty or wax-like materials with comparatively high proportions of water. These emulsions may be either water-in-oil (W/O) or oil-in-water (O/W) emulsions, depending primarily on the selection of the emulsifying agent. Such semisolid emulsions are also referred to as creams. Creams and ointments containing large amounts of insoluble powders are referred to as pastes. Pastes are usually stiffer and more absorptive than creams and ointments.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

The present invention includes a composition for treatment of hair which includes partially hydrolyzed fucoidan and a pharmaceutical carrier adapted for topical application to mammalian skin. The composition for treatment of hair of the present invention may be formulated to be applied to skin or hair, and more specifically to be applied to skin to prevent hair loss and/or promote hair growth.

Partially Hydrolyzed Fucoidan

The present invention advances prior art hair growth formulas by providing a hair growth formula formulated with fucoidan from seaweed, such as limu moui, kombu, or mozuku. The addition of fucoidan to the hair growth formula of the present invention serves to provide significant advantages not found in prior art hair growth formulas. The fucoidan-enhanced hair growth formulas of the present invention provides many beneficial functions, including providing for anti-aging, and regeneration of cells and tissues; promoting youthfulness; reducing inflammation and the like. In addition, the fucoidan-enhanced hair growth formulas of the present invention minimize the visible signs of both biological and environmental aging. That is, the present dietary supplements slow the aging process, assist in regenerating damaged cells and tissues, and promote growth factors in the body. Fucoidan is high in antioxidants that help to fight free radical damage to the body that may lead to cancer. These antioxidants help to fight free radical damage caused by the sun and other changing environmental conditions and elements.

Brown seaweed, a source of fucoidan, grows in many oceans, including off the coasts of Japan and Okinawa, Russian coastal waters, Tonga, and other places. An excellent source of fucoidan is the limu moui sea plant growing in the waters of the Tongan islands. This brown seaweed contains many vitamins, minerals, and other beneficial substances and is particularly rich in fucoidan.

Typically, the brown seaweed grows in long angel hair stems with numerous leaves. The fucoidan component is found in natural compositions on the cell walls of the seaweed, providing a slippery sticky texture that protects the cell walls from the sunlight.

In one embodiment, a kombu-type or mozuku-type seaweed is harvested from the coastal waters of the Tongan islands. These seaweeds can be manually harvested, including stems and leaves, by divers and cleaned to remove extraneous materials. The seaweed is then usually frozen in large containers and shipped to a processing plant.

In processing, the heavy outer fibers must first be broken down to provide access to the fucoidan component. If frozen, the seaweed material is first thawed. Then the seaweed material is placed in a mixing vat and shredded, while being hydrolyzed with acids and water. The material may be sulfonated with sulfuric acid to help in breaking down the heavy cell fibers. The mixture is also buffered with citric acid and thoroughly blended to maintain suspension. The material may also be heated at atmospheric or greater than atmospheric pressure while mixing. The resulting puree is tested and maintained at a pH of about 2 to 4 so as to remain acidic, enhancing preservative and stability characteristics.

The puree may be used in preparing the hair growth formula. Alternately, the mixture may be frozen in small containers for later processing. In another embodiment, the puree may be dehydrated, either partially, mostly, or completely, for use in the hair growth formula. Any technique for dehydrating slurries may be used, such as, freeze drying, spray drying, vacuum drying, and so forth.

An embodiment of the present invention provides hair growth formula formulated with fucoidan compositions from seaweed, such as the limu moui seaweed plant, the Japanese mozuku seaweed, or Japanese kombu seaweed, or mixtures thereof. According to another embodiment, the fucoidan may be partially hydrolyzed fucoidan. In yet another embodiment, the fucoidan may be sulfonated. In still another embodiment, the fucoidan compositions are present in selected embodiments in the amount of at least about 0.05 weight percent, or at least about 3 weight percent, or at least about 5 weight percent; and less than about 99 weight percent, or less than about 80 weight percent, or less than about 50 weight percent of the total weight of the composition.

In a further embodiment, the partially hydrolyzed fucoidan may be derived from Tongan limu moui, Japanese hoku kombu (Laminaria japonica), wakame, or mozuku (Cladosiphon and Nemacystus). The partially hydrolyzed fucoidan may be sulfonated.

Pharmaceutical Carrier

The pharmaceutical carrier of the present invention may be any capable of delivering the fucoidan to the skin to which the composition may be applied. Generally, the pharmaceutical carrier may be anhydrous or aqueous. The pharmaceutical carrier of the present invention may include water and/or one or more cosmetically acceptable organic solvents, and/or a powder or dry carrier. In addition to, or in place of the above-mentioned pharmaceutical carriers, a base may be used as the pharmaceutical carrier, or used along with the carriers mentioned above.

1. Pharmaceutical Carriers—Solvents

A solvent may be chosen from the group consisting of hydrophilic organic solvents, lipophilic organic solvents, amphiphilic solvents, or mixtures thereof. In addition to, or along with the water and/or solvents, a base may serve as the pharmaceutical carrier.

An anhydrous pharmaceutical carrier is generally one containing less than 1% by weight of water. This type of carrier may be an organic solvent, or even a dry/powder carrier. Some generally acceptable anhydrous pharmaceutical carriers includes organic solvents such as C₁-C₈ alcohols such as ethanol; alkylene glycols such as propylene glycol; alkyl ethers of alkylene glycols or of dialkylene glycols, in which the alkyl or alkylene radicals contain from 1 to 8 carbon atoms. Some examples of such solvents that are hydrophilic include ethanol, propanol, butanol, isopropanol, isobutanol, oxyethylenated polyethylene glycols, polyols, such as isoprene glycol, butylenes glycol, glycerol, sorbitol and its derivatives, glycol ethers, propylene glycol ethers, and mixtures thereof.

Some examples of amphiphilic organic solvents include polyols such as propylene glycol derivatives.

Some examples of lipophilic organic solvents include fatty esters.

Solvents are described in U.S. Pat. Nos. 4,139,619 and 4,596,812 both to Chidsey III, and both of which are herein incorporated by a reference.

When a solvent is used as the pharmaceutical carrier, either the fucoidan slurry or the dehydrated fucoidan may be added to the solvent to make the composition. Once the fucoidan is added to the solvent, it may be mixed by any means known in the art until it is substantially uniform.

2. Pharmaceutical Carriers—Powders

As to some of the powder-type pharmaceutical carriers, several are available. One such carrier typically used is talc. Useful talcs include hydrated magnesium silicates In one or more embodiments, talc can be represented by the formulae Mg₃Si₄O₁₀(OH)₂ or 3MgO.4SiO₂.H₂O. Exemplary forms of talc include talcum, soapstone, steatite, cerolite, magnesium talc, steatite-massive, and mixtures thereof. Talc filler may contain various other minerals such as dolomite, chlorite, quartz, and the like. Talc used as filler may also exhibit characteristics such as hydrophobicity, organophilicity, non-polarity, and chemically inertness. A representative talc is Talc 9107, which is available from Polar Minerals (Mt. Vernon, Ind.), which is non-abrasive, chemically inert, has a specific gravity of about 2.8, a pH of about 8.7, a refractive index of about 1.57 at 23° C., and a moisture content of less than about 0.3 weight percent.

Another suitable talc is Mistron Vapor Talc, which is available from Luzenac America (Centennial, Colo.). Mistron Vapor Talc is a soft, ultra-fine, white platy powder having a specific gravity of 2.75, a median particle size of 1.7 microns, an average surface area of 18 m²/g, and a bulk density (tapped) of 20 lbs/ft³. Other talc available from Luzenac America (Centennial, Colo.), includes Vertal MB, and Silverline 002. In one embodiment, talc is characterized as a platy, chemically inert filler having a specific gravity of from about 2.6 to about 2.8, a pH of about 7, and a moisture content of less than about 0.5 weight percent.

Another suitable powder pharmaceutical carrier includes bentonite. Bentonite is aluminum phyllosilicate and also consists of impurities such as minerals in the montmorillonite group, and may include minor amounts of minerals in the smectite group. Bentonite expands when wet. Sodium bentonite can absorb several hundred percent of its dry weight in water. Pure grades of bentonite may be edible. One example of edible bentonite is sold in its hydrated form by 1001 Herbs (Aurora, Colo.).

Other suitable powder pharmaceutical carriers include hydrated aluminum silicates. In one or more embodiments, useful clays can be represented by the formula Al₂O₃SiO₂.XH₂O. Exemplary forms of clay include kaolinite, montmorillonite, atapulgite, illite, bentonite, halloysite, and mixtures thereof. In one embodiment, the clay is represented by the formula Al₂O₃SiO₂.3H₂O. In another embodiment, the clay is represented by the formula Al₂O₃SiO₂.2H₂O. In one embodiment, the clay has a pH of about 7.0.

In one or more embodiments, various forms or grades of clays may be employed. Exemplary forms or grades of clay include air-floated clays, water-washed clays, calcined clays, and chemically modified (surface treated) clay.

Air-floated clays include hard and soft clays. In one or more embodiments, hard clays include those characterized as having a lower median particle size distribution, and higher surface area than soft clays. In one or more embodiments, soft clays include those characterized by having a higher median particle size distribution and lower surface area than hard clays. Hard and soft clays are disclosed in U.S. Pat. Nos. 5,468,550, and 5,854,327, which are incorporated herein by reference.

In one embodiment, the air-floated clays used have a pH of from about 4.0 to about 8.0, and in another embodiment, the pH is about neutral. The airfloated clays have an average particle size of less than about 2 microns. Typical airfloated clays have a specific gravity of around 2.6 g/cc.

Airfloated clays, both hard and soft, are available through various sources. Available from Unimin Corporation (New Canaan, Conn.) is Snobrite AF, which is an airfloated hard clay having a pH of about 5.5 to 7.5, a median particle size of about 1 micron, and a specific gravity of about 2.6 g/cc. Available from Kentucky-Tennessee Clay Company (Mayfield, Ky.) is Paragon, which has a pH of about 4.5 to 5.5, a median particle size of about 1 micron, and a specific gravity of about 2.6 g/cc, a soft airfloated clay from Unimin Corporation (New Canaan, Conn.) is Hi White R®, which has a pH of about 6.25, a median particle size of less than about 1 micron, and a specific gravity of about 2.6 g/cc, Alumex, and Suprex, all airfloated soft clays. Available from J.M. Huber Corporation (Atlanta, Ga.) is Barden R, and LGB, which are both airfloated hard clays, and K-78, an airfloated soft clay. Available from R.T. Vanderbilt Company (Norwalk, Conn.) is McNamee Clay, which is an airfloated soft clay having a pH of about 5.0 to 7.5, a median particle size of about 1 micron and a specific gravity of about 2.6 g/cc.

Water washed clays include those clays that are more closely controlled for particle size by the water fractionation process. This process permits the production of clays within controlled particle size ranges. In one embodiment, the average particle size of the clay is less than about 2 microns in diameter. In another embodiment, the pH of the clay is about 7. Available from J.M. Huber Corporation (Atlanta, Ga.) are water washed clays such as Polyfil® DL, Polyfil® F, Polyfil® FB, Polyfil® HG-90, Polyfil® K and Polyfil® XB. In one embodiment, a water washed kaolin clay includes hydrated aluminum silicate and titanium dioxide, which has a pH of from about 6 to about 7.5, and a specific gravity of about 2.6 g/cc.

Calcined clays include those that result from the removal of water contained in clays (clays typically contain about 14 percent water) by calcinations. The amount of bound water removed determines the degree of calcinations. In one embodiment, the average particle size of the clay is less than about 2 microns in diameter. In another embodiment, the pH of the clay is about 7. Available from J.M. Huber Corporation (Atlanta, Ga.) are calcined clays such as Polyfil® 40, Polyfil® 70, and Polyfil® 80.

Chemically modified (surface treated) clays include those that have. Crosslinking ability, which can be is imparted to the clay by modifying the surface of individual particles with a polyfunctional silane coupling agent. In one embodiment, the average particle size of the clay is less than about 2 microns in diameter. In another embodiment, the pH of the clay is about 7. Available from J.M. Huber Corporation (Atlanta, Ga.) are Nucap® 100 G, Nucap® 200, Nucap® 190, Nucap® 290, Nulok® 321, Nulok® 390, and Polyfil® 368.

Micas may also be used as the powdered pharmaceutical carrier. Mica includes mixtures of sodium and potassium aluminum silicate. Mica can be defined by the chemical formula αΔ₂₋₃(Ω)₄O₁₀(Σ)₂, where the α ion is potassium, sodium, barium, calcium, cesium, and/or ammonium, the Δ ion is aluminum, lithium, iron, zinc, chromium, vanadium, titanium, manganese, and/or magnesium, the Ω ion is silicon, aluminum, beryllium, boron, and/or iron (+3), and Σ is oxygen, fluorine, or hydroxide ion. Micas include true micas, brittle micas, and interlayer-deficient micas. True micas include a majority of singularly charged ions (e.g., potassium and sodium) in the α position. Brittle micas include a majority of doubly charged ions (e.g., calcium or barium) in the α position. Interlayer-deficient micas include fewer cations in the interlayer (the layer between the tetrahedral-octahedral-tetrahedral layers of the crystalline structure) than true or brittle micas.

Examples of true micas include aluminoceladonite (potassium aluminum magnesium iron silicate hydroxide), boromuscovite (potassium boro-silicate hydroxide), celadonite (potassium iron magnesium silicate hydroxide), chromphyllite (potassium chromium aluminum silicate hydroxide fluoride), ferro-aluminoceladonite (potassium aluminum iron magnesium silicate hydroxide), ferroceladonite (potassium iron magnesium silicate hydroxide), muscovite (potassium aluminum silicate hydroxide), nanpingite (cesium aluminum silicate hydroxide), paragonite (sodium aluminum silicate hydroxide), roscoelite (potassium vanadium aluminum silicate hydroxide), tobelite (ammonium aluminum silicate hydroxide), annite (potassium iron aluminum silicate hydroxide), aspidolite (sodium magnesium aluminum silicate hydroxide), biotite (potassium magnesium iron aluminum silicate hydroxide fluoride), eastonite (potassium magnesium aluminum silicate hydroxide), ephesite (sodium lithium aluminum silicate hydroxide), hendricksite (potassium zinc aluminum silicate hydroxide), lepidolite (potassium lithium aluminum silicate fluoride hydroxide), masutomilite (potassium lithium aluminum manganese silicate fluoride), montdorite (potassium iron manganese magnesium aluminum silicate fluoride), norrishite (potassium lithium manganese silicate), polylithionite (potassium lithium aluminum silicate fluoride), phlogopite (potassium magnesium aluminum silicate hydroxide), preiswerkite (sodium magnesium aluminum silicate hydroxide), siderophyllite (potassium iron aluminum silicate hydroxide), tainiolite (potassium lithium magnesium silicate fluoride), tetra-ferri-annite (potassium iron silicate hydroxide), tetra-ferriphlogopite (potassium magnesium iron silicate hydroxide), trilithionite (potassium lithium aluminum silicate fluoride), zinnwaldite (potassium lithium iron aluminum silicate fluoride hydroxide), and mixtures thereof.

Examples of brittle micas include chernykhite (barium vanadium aluminum silicate hydroxide), margarite (calcium aluminum silicate hydroxide), anadite (barium potassium iron magnesium aluminum silicate hydroxide), bityite (calcium lithium aluminum beryllium silicate hydroxide), clintonite (calcium magnesium aluminum silicate hydroxide), kinoshitalite (barium magnesium aluminum silicate hydroxide), and mixtures thereof.

Examples of interlayer deficient micas include brammallite (sodium aluminum silicate hydroxide), glauconite (potassium sodium iron aluminum magnesium silicate hydroxide), illite (potassium pluminum silicate hydroxide), wonesite (sodium magnesium aluminum silicate hydroxide), and mixtures thereof.

Other pharmaceutical carriers that are in powder form that may be used include chalk, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

When a powder is used as the pharmaceutical carrier, the fucoidan may be in either the dehydrated form, or in the slurry form. In one embodiment, the fucoidan may be substantially completely dehydrated and powdered. The dehydrated and powdered fucoidan may be mixed with the powdered pharmaceutical carrier by any means known in the art of mixing powders until it is substantially uniform.

In another embodiment, the slurry form of the fucoidan is used with the powder. In this embodiment, the slurry may be added with the powder to form a more viscous paste, (depending on the water content of the slurry) as the powder may adsorb a portion of the water from the fucoidan slurry. This may be a useful embodiment where the composition is meant to be applied to skin that is typically vertical relative to the ground, so that it adheres well to the skin without falling off.

3. Bases

Ideally, an ointment base should be nonirritating, nondehydrating, nongreasy, compatible with active components, stable, easily removable with water, absorptive (able to absorb water and/or other liquids), and able to efficiently release the incorporated active components. Ointments may be classified according to type, based on composition. Such ointment classes include oleaginous bases, absorption bases, emulsion bases, and water-soluble bases.

Oleaginous bases are generally anhydrous, hydrophobic, insoluble in water, and are not water-removable. Oleaginous bases includes the early ointments, which consisted almost entirely of vegetable and animal fats, as well as petroleum hydrocarbons. Fixed oils of vegetable origin include olive, cottonseed, sesame, persic, and other oils. Hydrocarbon bases include ointments prepared from petrolatum or liquid petrolatum with wax or other stiffening agents. Hydrocarbon bases do not become rancid, which is an advantage compared to animal fats and vegetable oils. Another oleaginous base includes silicones, which are synthetic polymers in which the basic structure is an alternating chain of silicon and oxygen atoms (e.g., —O—Si—O—Si—O—Si—). Silicones used in the pharmaceutical and cosmetic industries include dimethylpolysiloxane, methylphenylpolysiloxane, and a stearyl ester of dimethylpolysiloxane, all of which are insoluble in water and are water repellant. Illustrative oleaginous bases are well known in the art, such as Silicone Gibson Base and Vanisil Silicone Ointment.

Absorption bases are generally anhydrous, hydrophilic, insoluble in water, and most are not water-removable. These bases have the property of absorbing several times their weight of water and forming emulsions while retaining their ointment-like consistency. Absorption bases vary in their composition, but for the greater part, they are mixtures of animal sterols with petrolatum. Combinations of cholesterol and/or other lanolin fractions with white petrolatum are such absorption bases, and Eucerin® and Aquaphor® (a registered trademark of, and available from Beirsdorf Aktiengesellschaft Corporation, Germany) were among the earliest commercial bases of this type. Zopf Emollient Cream, Hoch Formula, Hydrophilic Petrolatum Base, Wool Alcohols Base, and Aquabase Ointment are absorption bases described herein. Some commercially available absorption bases include Polysorb (made by Fougera, a division of Altana Inc, Melville, N.Y.), and Nivea® Cream (registered trademark of, and made by Duke Laboratories, South Norwalk, Conn.).

Emulsion bases may be either W/O bases, which are hydrous, insoluble in water, and not removable with water and will absorb water, or O/W bases, which are hydrous, insoluble in water, and water-removable and will absorb water. These preparations are solid emulsions, and similar products have long been used as cosmetic creams. The availability of numerous compounds for use as wetting agents, dispersing agents, emulsifiers, penetrants, emollients, detergents, hardeners, preservatives, and the like has given a great deal of flexibility to ointment formulation. Although surface-active agents (i.e., surfactants) may be ionic or nonionic, the nonionic agents are widely used in dermatologic and pharmaceutical preparations. Polysorbate 80 (e.g., Tween 80) and Polyoxyl 40 Stearate represent such surfactants. Nonionic surfactants are generally less toxic and less irritating than ionic surfactants. Other advantages include their virtual neutrality, stability to freezing, stability to electrolytes, and ease of use. In general, the emulsion bases contain an aqueous phase, an emulsifying agent, and an oleaginous phase. The water phase of illustrative emulsion bases typically varies from 10 to 80% by weight of the total base. Glycerin, propylene glycol, or a polyethylene glycol is generally included with the aqueous phase to serve as a humectant, to reduce water loss through evaporation, and to lend a general softness to the creams. The addition of certain alcohols to emulsion base formulas also adds stability to the emulsion and imparts a smooth feel to the skin. Stearyl alcohol, a solid, increases the consistency of the ointment and permits the incorporation of more liquid components. Due to their ability to become hydrated, such alcohols assist in water retention of emulsion bases. The oleaginous phase may contain one or more of the following or similar components: petrolatum, fats, waxes, organic alcohols, polyglycol esters, or other grease-like substances. These substances are emulsified with the aqueous phase through the action of the surfactant. A few such emulsifiers include alkali soaps, alkyl sulfates, amine soaps, polyglycol esters, alkyl aryl sulfates, quaternary ammonium compounds, and the like. These emulsifying compounds aid in the dispersion of the fats and waxes in water and increase the stability of the ointments. Hydrophilic Ointment Base, Beeler's Base, and U.C.H. Base are illustrative O/W emulsion bases described herein. Commercially available O/W emulsion bases include Cetaphil® Cream (registered trademark of, and made by Galaderma Laboratories, L.P., Princeton, N.J.), Neobase (made by Neobase, Seattle, Wash.), Unibase® (registered trademark, made by Pfizer, New York, N.Y.), Dermovan, Phorsix Cream, Lubriderm® Cream (registered trademark, made by Pfizer, New York, N.Y.), and Velvachol® (registered trademark, available from Galderma Laboratories, Inc., Fort Worth, Tex.).

Water-soluble bases are anhydrous, soluble in water, water-removable, and greaseless, and will absorb water. These bases include those bases prepared from polyethylene glycols as well as semisolid preparations containing bentonite, colloidal magnesium aluminum silicate, and sodium alginate. Polyethylene glycol (PEG) compounds 1500, 1540, 4000, and 6000 are of interest in ointment and lotion formulations. PEG 1500 is a soft waxy solid, similar in consistency to petrolatum, with a congealing range of 40° C. to 45° C. PEG 1540 is a solid of consistency of beeswax and is intermediate in physical properties between the 1500 and 4000 PEGs. PEG 4000 has a congealing range of 53° C. to 56° C. and is most useful as a component of being an ointment base for, in addition to the general property of being an emulsifying and dispersing agent, it also adds to the consistency of the base. Both PEG 4000 and PEG 6000 are nonhygroscopic. PEG 6000 is a hard, translucent, waxy solid, and has a congealing range of 58° C. to 62° C.

Glyceryl monostearate is a polyhydric alcohol ester that has been widely used in cosmetic and ointment bases. It has a high melting point (56° C. to 58° C.) and is a good emulsifying agent. Glyceryl monostearate emulsions generally contain high water phases, usually above 60% by weight. It has the disadvantage of being incompatible with acids. Glyceryl Monostearate Base is described herein.

Cellulose derivatives, such as methylcellulose and hydroxyethyl cellulose, form colloidal solutions that resemble gums and mucilages, but are not as vulnerable to fungal or bacterial attack. Methylcellulose is dispersible in cold water, but in concentrated solutions will coagulate upon heating. Hydroxyethyl cellulose is more soluble at elevated temperatures so that viscosity of aqueous solutions decreases slightly on warming. It is a good protective colloid for aqueous dispersions of oils, waxes, and pigments. Sodium carboxymethylcellulose is another cellulose derivative frequently referred to as carboxymethyl cellulose or CMC. It is an anionic compound and thereby may be used as a thickening or stabilizing agent for suspensions and for ointments of the emulsion type where the emulsifying agent is anionic or nonionic. Any of these cellulose derivatives may be used to stabilize ointment formulas, and they are commercially available in various viscosity types and with various degrees of substitution.

Sodium alginate is a hydrophilic colloid that is compatible with small amounts of alcohol, glycerin, polyglycols, wetting agents, and solutions of alkali carbonates. It functions satisfactorily under acid or alkaline conditions within the pH range of 4.5-10. It is possible to make sodium alginate solutions into semi-firm or firm gels by the addition of small amounts of soluble calcium salts, i.e., calcium gluconate, calcium tartrate, and calcium citrate. Ions of the alkaline earth metals will thicken or gelatinize sodium alginate solutions when present in low concentrations, while at high concentrations they will precipitate them. A 2.5% solution of sodium alginate is a satisfactory inert diluent for greaseless and other types of ointments.

Bentonite, a colloidal hydrated aluminum silicate, is insoluble in water, but when mixed with 8 to 10 parts of water it swells to produce a slightly alkaline gel resembling petrolatum. The consistency of the product may be regulated by varying the amounts of water added. Ointments prepared from bentonite and water alone are found to be slightly drying and unstable upon standing, but addition of a humectant, such as glycerin or sorbitol, in amounts up to about 10% by weight will retard this action. Ointments prepared from bentonite do not encourage mold growth, and they have the advantage of not spreading to the hair when applied to the scalp.

Colloidal magnesium aluminum silicate (e.g., Veegum®, registered trademark of, and available from R.T. Vanderbilt Company, Inc.) is an inorganic emulsifier, suspending agent, and thickener. Dispersions are slightly alkaline and are compatible with about 20 to 30% ethyl alcohol, isopropyl alcohol, acetone, and similar solvents. Glycols, such as glycerin and propylene glycol, are compatible at 40 to 50% concentrations.

Carbopol® 934 (carboxypolymethylene, registered trademark of, and made by B. F. Goodrich Chemical Co., Akron, Ohio) is an acid polymer that disperses readily in water to yield an acid solution of low viscosity. When the acid solution is neutralized with a suitable base, such as sodium bicarbonate, sodium hydroxide, or the like, a clear, stable gel results. Carbopol® 934 is inert physiologically and is neither a primary irritant nor a sensitizer. The thickening efficiency of Carbopol® 934 may be used in the preparation of such pharmaceuticals as creams, ointments, lotions, suspensions, and emulsions.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

Additional Components

The hair treatment compositions of the present invention may include additionale ingredients. Among these components include compounds that are disclosed to have the effect of slowing hair loss and/or promoting hair growth. Some examples of these compounds include, for example, 6-amino-1,2-dihydro-1-hydroxy-2-imino-4-piperidinopyrimidine, 4-(O-methyloxime)phenyl ester of 2-hydroxy-4-(3,3,3-trifluoro-2-hydroxy-2-methylpropionylamino)benzoic acid; phenyl ester of 2-hydroxy-4-(3,3,3-trifluoro-2-hydroxy-2-methylpropionylamino)benzoic acid; methyl ester of 4-(3,3,3-trifluoro-2-hydroxy-2-methylpropionylamino)benzoic acid; para-tolyl ester of 2-hydroxy-4-(3,3,3-trifluoro-2-hydroxy-2-methylpropionylamino)benzoic acid; phenyl ester of 2-hydroxy-4-(2-phenyl-2-hydroxypropionylamino)benzoic acid; methyl ester of 2-methoxy-4-(3,3,3-trifluoro-2-hydroxy-2-methylpropionylamino)benzoic acid; phenyl ester of 2-chloro-4-(3,3,3-trifluoro-2-hydroxy-2-methylpropionylamino)benzoic acid; methyl ester of 2-ethyl-4-(3,3,3-trifluoro-2-hydroxy-2-methyl propionylamino)benzoic acid; 3,5-bis(trifluoromethyl)benzyl ester of 2-hydroxy-4-(3,3,3-trifluoro-2-hydroxy-2-methylpropionylamino)benzoic acid; tert-butyl ester of 2-hydroxy-4-(3,3,3-trifluoro-2-hydroxy-2-methylpropionylamino)benzoic acid; 3-morpholinopropyl ester of 2-hydroxy-4-(3,3,3-trifluoro-2-hydroxy-2-methylpropionylamino)benzoic acid; 4-octylphenyl ester of 2-hydroxy-4-(3,3,3-trifluoro-2-hydroxy-2-methylpropionylamino)benzoic acid; 2,4-diaminopyrimidine-N-oxide, methyl ester of 2-hydroxy-4-(3,3,3-trifluoro-2-hydroxy-2-methylpropionylamino)benzoic acid, nicotinic acid esters, including in particular tocopherol nicotinate, benzyl nicotinate and C₁-C₁ alkyl nicotinates such as methyl or hexyl nicotinates; calcium-antagonizing agents such as cinnarizin, diltiazem, nimodipine and nifedipine; hormones such as estriol and its analogs, or thyroxin and its salts; steroidal or nonsteroidal anti-inflammatory agents, such as corticosteroids (for example: hydrocortisone); antiandrogenic agents such as oxendolone, spironolactone, diethylstilbestrol and flutamide; steroidal or nonsteroidal inhibitors of 5-.alpha.-reductases such as finasteride; potassium agonists such as cromakalim and nicorandil; pyrimidine derivatives, such as 2,4-diamino-6-piperidinopyrimidine 3-oxide, diazoxide, spiroxasone, phospholipids such as lecithin, linoleic and linolenic acids, salicylic acid and its derivatives hydroxycarboxylic or ketocarboxylic acids and their esters, lactones and their corresponding salts, anthralin, carotenoids, eicosatetraenoic and eicosatrienoic acids or their esters and amides, vitamin D and its derivatives, ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, and the 7,9(11)-dien-6-one ecdysteroids. Ecdysteroids are discussed in detail in U.S. Patent Application Publication No. 2005/0137175, which is herein incorporated by a reference.

The composition may further include anti-wrinkle compounds. Exemplary anti-wrinkle compounds suitable for use in the compositions of the present invention include hydroxy acids (e.g., salicylic acid, glycolic acid), keto acids (e.g., pyruvic acid), ascorbic acid (vitamin C) and its derivatives, phytic acid, lysophosphatidic acid, flavonoids (e.g., isoflavones, flavones, etc.), stilbenes, cinnamates, resveratrol, kinetin, zeatin, dimethylaminoethanol, peptides from natural sources (e.g., soy peptides), salts of sugar acids (e.g., Mn gluconate), and retinoids which enhance the keratinous tissue appearance benefits of the present invention, especially in regulating keratinous tissue condition, e.g., skin condition, and other vitamin B compounds (e.g., thiamine (vitamin B1), pantothenic acid (vitamin B5), carnitine (vitamin Bt), riboflavin (vitamin B2), and their derivatives and salts (e.g., HCl salts or calcium salts)), and other compounds as herein described. These may be in their natural form or in a synthetically-produced form. These may be purified or used in an impure state.

Another type of compound to use as an anti-wrinkle compound include substances having an epithelium-abrasive action such as α-hydroxy acids including lactic acid and glycolic acid and β-hydroxy acids represented by salicylic acid. Also amino acids, polyhydric alcohols, polysaccharides, lipids such as ceramides may be used.

In one embodiment, the present composition includes anti-oxidants. The amount of anti-oxidants of the present invention may be from about 0.01 weight percent to about 10 weight percent. Some examples of anti-oxidants may include tocopherol (vitamin E), tocopherol sorbate, tocopherol acetate, other esters of tocopherol, 6-hydroxy-2,5,7,8-tetramethylchroman-2-carboxylic acid (commercially available under the tradename Trolox.sup.R), amines (e.g., N,N-diethylhydroxylamine, amino-guanidine), nordihydroguaiaretic acid, bioflavonoids, amino acids, silymarin, tea extracts, and grape skin/seed extracts may be used.

In one embodiment, the composition of the present invention includes dehydroacetic acid, its isomers, derivatives, tautomers, and/or pharmaceutically acceptable salts thereof. As used herein, “pharmaceutically acceptable” means that the salts of dehydroacetic acid are suitable for use in contact with the tissues of mammals to which they will be exposed without undue toxicity, incompatibility, instability, irritation, allergic response, and the like. The technical name for dehydroacetic acid is 3-Acetyl-6-methyl-2H-pyran-2,4(3H)-dione and can be commercially purchased from Lonza Group, Ltd. Of Basel, Switzerland.

Pharmaceutically acceptable salts include alkali metal salts, such as sodium and potassium; alkaline earth metal salts, such as calcium and magnesium; non-toxic heavy metal salts; ammonium salts; and trialkylammonium salts, such astrimethylammonium and triethylammonium. Sodium, potassium, and ammonium salts of dehydroacetic acid are useful. Highly useful is sodium dehydroacetate which can be purchased from Tri-K, as Tristat SDHA. Derivatives of dehydroacetic acid incude, but are not limited to, any compounds wherein the CH₃ groups are individually or in combination replaced by amides, esters, amino groups, alkyls, and alcohol esters. Tautomers of dehydroacetic acid are the isomers of dehydroacetic acid which can change into one another with great ease so that they ordinarily exist in equilibrium. Thus, tautomers of dehydroacetic acid can be described as having the chemical formula C₈H₈O₄ and generally having the structure above.

In one embodiment, the present composition includes flavonoids. Flavonoids may be incorporated in the hair growth formula in the amount of from about 0.01 to about 20 weight percent. Flavanones such as unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents. Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

In another embodiment, the composition may include conditioning agents. Conditioning agents of the present embodiment may include humectants, moisturizers, skin conditioners, and so forth. The amount of these conditioning agents may be from about 0.01 weight percent to about 20 weight percent. The conditioning agents may include, for example, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof.

Further, components formulated to improve the collagen and/or elastin of the skin may be added. U.S. Pat. No. 6,641,848, which is herein incorporated by reference, discloses formulas for increasing collagen IV. Collagen is a major constituent of the junction between the dermis and the epidermis. Certain compounds have been found to strengthen this junction, which is required for proper functioning of the skin. Some of these compounds include, for example, triterpenic saponins and sapogenols. Saponins or sapogenols may be extracted from plants, such as Glycine max (soya), Phaseolus vulgaris, Phaseolus aureus, Phaseolus lunatus, Vicia faba, Lens culinaris, Cicer arietum, Vigna angularis, Vigna mungo, Oxytropis ochrocephala, Oxytropis glabra, Pisum sativum, Sophora favescens, Asparalus membranaceus, Crotalaria albida, Arachis hypogea, Galega officinalis, Wistaria brachybotrys and Trifolium repens, or those extracted from plants of the Medicago type, particularly Medicago alfalfa and Medicago sativa, which is often called “alfalfa”. In another example, hydroxyprofisiland-C may be added.

According to yet another embodiment of the present invention, the hair growth formula includes antimicrobial and/or antifungal compounds. The amount of these compounds in the hair growth formula may be from about 0.001 to about 10 weight percent. Some suitable antimicrobial/antifungal compounds may include, for example, benzoyl peroxide, 3-hydroxy benzoic acid, glycolic acid, lactic acid, 4-hydroxy benzoic acid, 2-hydroxybutanoic acid, 2-hydroxypentanoic acid, 2-hydroxyhexanoic acid, phytic acid, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, ciclopirox, lidocaine hydrochloride, clotrimazole, miconazole, ketoconazole, neocycin sulfate, and mixtures thereof.

Some other useful natural components that may be used include, for example, witch hazel, mangosteen, honey, aloe, sage, piper, clove, ginger, red pepper, willow, rhubarb, sesame, chamomile, propolis, thyme, lavender, cinnamon oil, flower or blossom oils, olive oil, palm oil, coconut oil, beeswax, and so forth. One particularly beneficial natural component is a derivative of the mangosteen plant. According to one embodiment, the present invention includes from about 0.01 to about 10 weight percent of a derivative of the mangosteen plant.

The Mangosteen plant (Garcinia mangostana L.) is a tropical fruit-bearing plant named after the French explorer Laurent Garcin. Many of the benefits of the mangosteen plant and its derivatives are descrived in U.S. Pat. No. 6,730,333, which is herein incorporated by a reference. Over the years, the mangosteen plant has been used in a number of different ways. The timber is used for cabinets, building materials, fencing and furniture. The pericarp, containing pectin, tannins, resins and a yellow latex, is used in tanning and dyeing leather black. The fruit pulp is mostly used as a dessert, but can also be canned or made into preserves. However, when removing the fruit pulp from the rind, care must be taken to prevent the tannins and resins of the cut pericarp from contacting the fruit pulp. The mangosteen rind, leaves and bark have also been used as components in folk medicine in areas where the plant grows indigenously. The thick mangosteen rind is used for treating catarrh, cystitis, diarrhea, dysentery, eczema, fever, intestinal ailments, itch, and skin ailments. The mangosteen leaves arc used by some natives in teas and other decoctions for diarrhea, dysentery, fever, and thrush. It is also known that concoctions of mangosteen bark can be used for genitourinary afflictions and stomatosis.

Some of the medicinal properties of the Garcinia mangostana L. plant have been the subject of pharmacological and clinical studies. These studies have isolated chemical constituents in the mangosteen leaves, wood, pericarp and seed aril, which were found to contain the following biologically active compounds, among others: 1,6-dihydroxy-3-methoxy-2-(3-methyl-2-butenyl) xanthone, 1,5,8-trihydroxy-3-methoxy-2-(3-methyl-2-butenyl) xanthone, maclurin, 1,3,6,7-tetrahydroxy xanthone, 1,3,6,7-tetrahydroxy xanthone-O-β-D-glucoside, chrysanthemin, cyaniding-3-O-β-D-sophoroside, 8-deoxygartanin, 1,5-dihydroxy-2-isopentenyl-3-methoxy xanthone, 1,7-dihydroxy-2-isopentenyl-3-methoxy xanthone, 5,9-dihydroxy-8-methoxy-2,2-dimethyl-7-(3-methylbut-2-enyl)2(H), 6(H)-pyrano-(3,2,6)-xanthen-6-one, fructose, garcinone A, B, C, D and E, gartanin, glucose, cis-hex-3-enyl acetate, 3-isomangostin, 3-isomangostin hydrate, 1-isomangostin, 1-isomangostin hydrate, kolanone, mangostin, β-mangostin, α-mangostin, mangostin-3,6-di-O-gulcoside, normangostin, sucrose, tannins, BR-xanthone-A, BR-xanthone-B, calabaxanthone demethylcalabaxanthone, 2-(γ,γ-dimethylallyl)-1,7-dihydroxy-3-methoxyxanthone, 2,8-bis-(γ,γ-dimethylallyl)-1,3,7-trihydroxyxanthone, 1,3,5,8-tetrahydroxy-2,4-diprenylxanthone, and mangostanol. Many of these chemical constituents are xanthones, which are biologically active compounds that are receiving increasing interest in pharmacological studies for a variety of health benefits.

The hair growth formulas of the present invention may also contain fragrances, proteins, colorants or coloring agents, vitamins, botanical extracts, glycolipids, polymers, copolymers, and the like, as are generally known in the art of making skin care products. The Cosmetic, Toiletry, and Fragrance Association's International Cosmetic Component Dictionary and Handbook is an excellent source of information concerning such components.

As used herein, “colorants” or “coloring agents” are agents that give hair growth formulas a more pleasing appearance, and in addition help the manufacturer to control the product during its preparation and help the user to identify the product. Any of the approved certified water-soluble FD&C dyes, mixtures thereof, or their corresponding lakes may be used to color hair growth formulas. A color lake is the combination by adsorption of a water-soluble dye to a hydrous oxide of a heavy metal, resulting in an insoluble form of the dye.

The hair growth formulas of the present invention may include an analgesic. Analgesics are typically used to assist in the alleviation of pain. Some examples of the analgesics than may be included in the present hair growth formula include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, arncinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, piroxicam, isoxicam, tenoxicam, sudoxicam, CP-14,304, aspirin, disalcid, benorylate, trilisate, safapryn, solprin, diflunisal, and fendosal, diclofenac, fenclofenac, indomethacin, sulindac, tolmetin, isoxepac, furofenac, tiopinac, zidometacin, acematacin, fentiazac, zomepiract, clidanac, oxepinac, and felbinac, mefenamic, meclofenamic, flufenamic, niflumic, and tolfenamic acid, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic, phenybutezone, oxyphenbutezone, feprazone, azapropezone, and trimethazone and mixtures thereof.

The hair growth formulas of the present invention may include a radiation protecting agent. Damage to the skin may occur from ultraviolet radiation, in particular from the sun. This damage can be especially harsh for skin that is substantially perpendicular to the direction of the radiation, as the top of the head typically is. To stop or slow this damage, it may be necessary to block or absorb the radiation before it reaches and damages the skin. The radiation protecting agent may be any that is known in the art of sunblocks or sunscreens. Some of the major types of radiation protecting agents include physical blockers, and chemical protectants. Phyisical blockers work by physically blocking the solar radiation from reaching the skin. Some examples of theses physical blockers include titanium dioxide, aluminum oxide, magnesium dioxide, and zinc oxide, with titanium dioxide and zinc oxide being the most used in conventional sunblocks. Physical blockers typically block both UV-A and UV-B radiation from reaching the skin.

Chemical protectants include organic molecules with carbonyl groups. Some examples of chemical protectants include, for example, p-aminobenzoic acid (PABA), its salts and its derivatives (ethyl, isobutyl, glyceryl esters; p-dimethylaminobenzoic acid), anthranilates (i.e., o-aminobenzoates; 5, methyl, menthyl, phenyl, benzyl, phenylethyl, linalyl, terpinyl, and cyclohexenyl esters), salicylates (amyl, phenyl, benzyl, octyl, menthyl, glyceryl, and dipropyleneglycol esters), trolamine salicylate, avobenzone, cinnamic acid derivatives (methyl and benzyl esters, a-phenyl cinnamonitrile; butyl cinnamoyl pyruvate), cinoxate, dihydroxycinnamic acid derivatives (umbelliferone, methylumbelliferone, methylaceto-umbelliferone), trihydroxycinnamic acid derivatives (esculetin, methylesculetin, daphnetin, and the glucosides, esculin and daphnin), hydrocarbons (diphenylbutadiene, stilbene), dibenzalacetone, benzalacetophenone, dioxybenzone, naphtholsulfonates (sodium salts of 2-naphthol-3,6-disulfonic and of 2-naphthol-6,8-disulfonic acids), Dihydroxy-naphthoic acid and its salts, o- and p-hydroxybiphenyldisulfonates, homosalate, menthyl anthranilate, coumarin derivatives (7-hydroxy, 7-methyl, 3-phenyl), octyl methoxycinnamate (octinoxate), diazoles (2-acetyl-3-bromoindazole, phenyl benzoxazole, methyl naphthoxazole, various aryl benzothiazoles), quinine salts (bisulfate, sulfate, chloride, oleate, and tannate), quinoline derivatives (8-hydroxyquinoline salts, 2-phenylquinoline), hydroxy- or methoxy-substituted benzophenones, Uric and vilouric acids, tannic acid and its derivatives (e.g., hexaethylether), (Butyl carbotol) (6-propyl piperonyl) ether, hydroquinone, oxybenzone, padimate-o, phenylbenzimidazole sulfonic acid, benzophenones (oxybenzene, sulisobenzone, dioxybenzone, 2,2′,4,4′-tetrahydroxybenzophenone, 2,2′-dihydroxy-4,4′-benzoresorcinol, dimethoxybenzophenone, octabenzone), aminobenzoic acid, 4-isopropyldibenzoylmethane, butylmethoxydibenzoylmethane, etocrylene, 4-isopropyl-dibenzoylmethane, 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyidimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diph-enylacrylate, 2-ethylhexylsalicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-am-ino-benzoate, 2-phenylbenzimidazole-5-sulfonic acid, 2-(p-dimethylaminophenyl)-5-sulfonicbenzoxazoic acid, ensulizole, meradimate, and mixtures thereof. Many of these chemicals are regulated by the Food and Drug Administration as to the maximum amount that may be present in the composition for application to the hair or skin. For example, PABA is regulated such that the composition must not include more than 15% PABA. Further, many of the chemical protectants only protect against UV-A or UV-B. Some protect against both UV-A an UV-B, such as dioxybenzone, oxybenzone, and sulisobenzone.

The hair growth formulas of the present invention may include anti-oxidant or radical scavengers. Suitable anti-oxidants or radical scavengers include, but are not limited to, butylated hydroxy benzoic acids, 6-hydroxy-2,5,7,8-tetramethylchroman-2-carboxylic acid, gallic acid, propyl gallate, uric acid, sorbic acid, ascorbyl esters of fatty acids, amines, sulfhydryl compounds, dihydroxy fumaric acid, pharmaceutically acceptable salts thereof, alkyl esters thereof, derivatives thereof and mixtures thereof.

The hair growth formulas of the present invention may include topically administered vitamins. Such vitamins include, but are not limited to Vitamin A, ascorbic acid, Vitamin B, biotin, panthothenic acid, Vitamin D, Vitamin E and mixtures thereof and derivatives thereof. Derivatives or analogs of these vitamins may also be used such as synthetic Vitamin A analogs, natural Vitamin A analogs, geometric isomers and stereoisomers and mixtures thereof.

The hair growth formula may include propellants. Propellants may include propane, butane, isobutane, dimethyl ether, carbon dioxide, nitrous oxide.

The composition may include other additional components such as, for example, adjuvants, such as fatty substances, preserving agents, stabilizers, opacifiers, softeners, silicones, foaming agents, pH regulators, emulsifying agents, conventional hydrophilic or lipophilic thickeners and/or gelling agents, hydrophilic or lipophilic active aents, sequestering agents, polymers acidifying or basifying agents, fillers, ceramides, moisturizers, surfactants, anti-dandruff agents, or any of the adjuvant normally used in cosmetics. The amount of these adjuvants are those conventionally used in the field in question.

Thus, while the present invention has been fully described above with particularity and detail in connection with what is presently deemed to be the most practical embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A hair growth formula for topical application to the skin and/or hair, comprising partially hydrolyzed fucoidan and a pharmaceutical carrier.

2. The composition of claim 1, wherein the partially hydrolyzed fucoidan is derived from the group consisting of: Japanese mozuku seaweed, Japanese kombu seaweed, Tongan limu moui seaweed, and combinations thereof.

3. The composition of claim 1, wherein the partially hydrolyzed fucoidan is sulfonated.

4. The composition of claim 1, wherein the partially hydrolyzed fucoidan is dehydrated.

5. The composition of claim 4, wherein the pharmaceutical carrier comprises a powder.

6. The composition of claim 5, wherein the powder comprises mica.

7. The composition of claim 1, further comprising a radiation protecting agent.

8. The composition of claim 7, wherein the radiation protecting agent comprises titanium dioxide.

9. The composition of claim 1, further comprising a hair treatment component selected from the group consisiting of: 6-amino-1,2-dihydro-1-hydroxy-2-imino-4-piperidinopyrimidine, 4-(O-methyloxime)phenyl ester of 2-hydroxy-4-(3,3,3-trifluoro-2-hydroxy-2-methylpropionylamino)benzoic acid; phenyl ester of 2-hydroxy-4-(3,3,3-trifluoro-2-hydroxy-2-methylpropionylamino)benzoic acid; methyl ester of 4-(3,3,3-trifluoro-2-hydroxy-2-methylpropionylamino)benzoic acid; para-tolyl ester of 2-hydroxy-4-(3,3,3-trifluoro-2-hydroxy-2-methylpropionylamino)benzoic acid; phenyl ester of 2-hydroxy-4-(2-phenyl-2-hydroxypropionylamino)benzoic acid; methyl ester of 2-methoxy-4-(3,3,3-trifluoro-2-hydroxy-2-methylpropionylamino)benzoic acid; phenyl ester of 2-chloro-4-(3,3,3-trifluoro-2-hydroxy-2-methylpropionylamino)benzoic acid; methyl ester of 2-ethyl-4-(3,3,3-trifluoro-2-hydroxy-2-methylpropionylamino)benzoic acid; 3,5-bis(trifluoromethyl)benzyl ester of 2-hydroxy-4-(3,3,3-trifluoro-2-hydroxy-2-methylpropionylamino)benzoic acid; tert-butyl ester of 2-hydroxy-4-(3,3,3-trifluoro-2-hydroxy-2-methylpropionylamino)benzoic acid; 3-morpholinopropyl ester of 2-hydroxy-4-(3,3,3-trifluoro-2-hydroxy-2-methylpropionylamino)benzoic acid; 4-octylphenyl ester of 2-hydroxy-4-(3,3,3-trifluoro-2-hydroxy-2-methylpropionylamino)benzoic acid; 2,4-diaminopyrimidine-N-oxide, methyl ester of 2-hydroxy-4-(3,3,3-trifluoro-2-hydroxy-2-methylpropionylamino)benzoic acid, ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, 7,9(11)-dien-6-one ecdysteroid, and combinations thereof.

10. The composition of claim 1, further comprising an anti-oxidant.

11. The composition of claim 10, wherein the anti-oxidant comprises one of the group consisting of: butylated hydroxy benzoic acids, 6-hydroxy-2,5,7,8-tetramethylchroman-2-carboxylic acid, gallic acid, propyl gallate, uric acid, sorbic acid, ascorbyl esters of fatty acids, amines, sulfhydryl compounds, dihydroxy fumaric acid, pharmaceutically acceptable salts thereof, alkyl esters thereof, derivatives thereof and mixtures thereof.

12. The composition of claim 1, further comprising a flavonoid.

13. The composition of claim 12, wherein the flavonoid comprises one selected from the group consisting of: unsubstituted flavanone, mono-hydroxy flavanones, mono-alkoxy flavanones, unsubstituted chalcone, mono-hydroxy chalcones, di-hydroxy chalcones, tri-hydroxy chalcones, unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones, unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

14. The composition of claim 1, further comprising one natural component selected from the group consisting of: witch hazel, mangosteen, honey, aloe, sage, piper, clove, ginger, red pepper, willow, rhubarb, sesame, chamomile, propolis, thyme, lavender, cinnamon oil, flower or blossom oils, olive oil, palm oil, coconut oil, beeswax, and mixtures thereof.

15. The composition of claim 1, further comprising from about 0.01 weight percent to about 10 weight percent of a derivative of mangosteen.

16. The composition of claim 1, further comprising honey.

17. The composition of claim 1, further comprising an analgesic.

18. The composition of claim 17, wherein the analgesic is selected from the group consisting of: hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, arncinafide, betamethasone, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, piroxicam, isoxicam, tenoxicam, sudoxicam, CP-14,304, aspirin, disalcid, benorylate, trilisate, safapryn, solprin, diflunisal, and fendosal, diclofenac, fenclofenac, indomethacin, sulindac, tolmetin, isoxepac, furofenac, tiopinac, zidometacin, acematacin, fentiazac, zomepiract, clidanac, oxepinac, and felbinac, mefenamic, meclofenamic, flufenamic, niflumic, and tolfenamic acid, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic, phenybutezone, oxyphenbutezone, feprazone, azapropezone, and trimethazone and mixtures thereof.

19. The composition of claim 1, further comprising a base.

20. The composition of claim 19, wherein the base comprises an aqueous phase, an emulsifying agent, and an oleaginous phase.
